(19) United States
(12) Patent Application Publication
(10) Pub. No.: US 2005/0038400 A1
(43) Pub. Date: Feb. 17, 2005

(54) Leak prevention system for a disposable absorbent article

(76) Inventor: Simon K. Poruthoor, Madison, WI (US)

Correspondence Address: Roland W. Norris;Pauley Petersen Kinne & Erickson, Suite 365, 2800 West Higgins Road, Hoffman Estates, IL, 60195, US

(21) Appl. No.: 10/638,872

(22) Filed: Aug. 11, 2003

(51) Int. Cl. A61F 13/15; A61F 13/20

(52) U.S. Cl. 604/385.01; 604/387

(57) ABSTRACT

A disposable diaper includes a chassis having an interior leak prevention system of leg elastics, and desirably, containment flaps. The diaper further includes an exterior leak prevention system having straps refastenable to a locking area positioned at one of a crotch region and a more dorsal region of the garment. The straps do not elongate during normal wear and are connected between the crotch region and the waist region. Each strap provides a convenient and economical means of maintaining diaper position and skin contact of the diaper chassis interior leak prevention system against the wearer without placing tension on the chassis or redmarking the wearer.

### FIELD OF INVENTION

The present invention relates to disposable absorbent articles. More particularly, the present invention relates to a disposable diaper for an infant or an adult, including a leak prevention system that restricts movement of the diaper with respect to a skin surface of the wearer to prevent or reduce leakage of the diaper after insult.

### BACKGROUND OF THE INVENTION

In conventional absorbent articles, such as diapers and training pants, leakage of bodily exudates may occur around the legs of the wearer. Typically, this leakage occurs in areas of the diaper that are not closely aligned and/or in contact with a skin surface of the wearer. In order to minimize the undesirable leakage of bodily exudates, conventional diapers may include elastic components, such as containment flaps and/or leg elastic components, which form a “gasket” around the legs of the wearer when the diaper is properly positioned about the wearer. While the elastic components about the legs of the wearer generally prevent or decrease the occurrence of leakage, the tension of the elastic components may result in “red marking,” if the tension is too great. The term “red marking” refers to temporary marks on the skin surface of the wearer resulting from irritation of the skin. While a relatively lower tension may initially keep the diaper tightly positioned or fit about the wearer's legs, the positioning or fitting may not be close enough to prevent leakage, particularly after the diaper has been soiled and is subjected to movement of the wearer. When the diaper is insulted, i.e., soiled, or loaded with exudate, the increase in weight and/or expansion of the conventional diaper can cause the diaper to pull away from the legs of the wearer. Because the downward movement of the diaper with respect to the waist is not restricted, the pulling due to the weight and/or expansion of the diaper results in gaps or spaces formed between the wearer's legs and the diaper, potentially resulting in leakage.

Also known are various drawstrings arrangements such as discussed in European patent publication EP 1 011 582. Such drawstrings are used as supplements to, or in place of, leg elastics in most cases. Such drawstrings appear to require additional manufacturing operations and materials, leading to further expense of an absorbent garment, without distinctly affecting the direction of elastic forces already supplied with most of the known disposable garment types.

Accordingly, there remains a need for a diaper that provides a proper fit about the wearer's waist and legs, particularly after the diaper has been soiled, without irritating the wearer's skin. Further, there remains a need for a diaper component or system that constrains or restricts the undesirable movement of the diaper with respect to the skin surface due to increased weight and expansion of the diaper after the diaper has been soiled.

### SUMMARY OF THE INVENTION

In response to the difficulties and problems discussed above, the present invention provides a personal care garment, for convenience taught as an exemplary disposable diaper, having a mechanical support system which is configurable to redirect tension within, and reinforce the designed anti-leak aspects of, an ordinary personal care garment leak prevention system. The mechanical support system may comprise one or more straps running between two locations on the diaper exterior. The strap or straps (which, whether designed as generally unitary piece or two separate pieces, will be referred to generally herein in the plural for brevity) are desirably constructed of a material which does not elongate during normal wear and therefore places no tension on the diaper chassis or the body of the wearer during normal use. It will be appreciated that a consumer may fasten the straps so as to add tension to parts of the garment although such tensioning is not necessary for operation of the present invention. The straps may desirably be made of a non-extensible material, although in some embodiments a material which is extensible at forces above those normally expected from a loaded garment may be used. The term “non-extensible shall be used generically herein to refer to either of the above types of material. Desirably, the straps are easily and economically produced and added to exterior of the diaper.

A disposable diaper chassis generally defines a front waist region, a back waist region, and a crotch region that extends between and connects the front waist region and the back waist region covering the perineal area of the wearer. Left and right side panel regions generally cover the hip areas of the wearer. The diaper chassis will generally include an outer cover and a bodyside liner that is connected to the outer cover in a superposed relation. An absorbent core can be located between the outer cover and the bodyside liner. The absorbent core is configured to absorb and contain body exudates discharged from the wearer.

Generally, most modern diapers have an interior leak prevention system contained within the chassis which may include various elasticized boundaries around waist and leg openings, and often further includes so called “containment flaps” of barrier material designed to lie generally against the skin of the wearer and perpendicular to the flow of exudates towards the outside of the garment.

According to aspects of the present invention two straps, desirably, are positioned proximate to the laterally opposing side edges of the diaper in the crotch region and connected to, or integral with, the diaper outer cover. As stated above the straps are desirably made of a material that does not elongate during use, such as an inelastic and non-extensible material. However, in some embodiments an extensible or elastic material may be used consistent with the teachings of the present invention. The straps may have any suitable width and length respective to the chassis size. In one embodiment of this invention, the straps are permanently bonded proximate to the side edges of the diaper at the crotch region of the diaper. Alternatively, the straps may be permanently bonded at the back waist region or a dorsal half of a side panel region. In the case of permanent bonding at one end of the straps, the opposite ends are desirably left free with means for refastenable attachment to the exterior of the garment. Alternatively, both ends of a strap may be provided with means for refastenable attachment to the exterior of the garment. Permanent bonding of both ends of a strap to the garment exterior is less desirable.

The straps will generally extend between the crotch area of the diaper and the back waist region, with the back waist region being especially desirable for the placement of refastenable ends of the straps for practical reasons on infant garments. The straps may follow a course generally from the lateral margins of the crotch region, traversing the area of the side panel above the leg opening of the garment when transitioning from the front region of a garment to the back region of a garment and desirably terminating at a medial area of the back waist region although, as noted above, points of strap attachment may vary from the side panel region to the rear waist section. Generally, the straps will follow a path along the outside margins of the garment overlaying portions of the interior leak containment system, e.g., leg gasketing or elastics on the interior of the garment which are subject to leaks.

However, it will be understood by the person having ordinary skill in the art that such a strap placement is not for the purposes of providing additional tensioning to serve as a secondary leg-opening elastic or reinforcement thereof in the sense of known garments, but is merely an alternative route of force redirection against a loaded diaper according to the present invention.

The outer cover of a diaper according to aspects of the invention may include a locking area for releasable attachment of a first end of a refastenable strap end to be positioned at one of a crotch region or a back waist region of the diaper. Desirably the locking area may be integrated into the material of the outer cover whereby the strap may be releasably engaged anywhere on the outer cover. Alternatively, a specific area of the outer cover may be fitted with a cooperative fastener component designed to refastenably engage the end of a strap. A second strap may be independently provided or may diverge from the first strap to provide a second point of attachment and means of supporting the interior leak prevention system of the second leg. In an exemplary embodiment, two individual straps will generally extend in substantially symmetrical courses on either side of the body/garment. The straps are desirably easily attached and repositioned so as to adjust the load carrying capability and support for the containment features within the diaper chassis, as necessary to hold leakage to a minimum.

Desirably, a first strap is positionable above at least a portion of a first leg opening formed by the diaper chassis and is refastenably engageable with the locking area. For example, the first strap may be connected to the front outer cover of the diaper at the crotch region and extend about at least a portion of the chassis towards the back waist area. Desirably, the first strap extends along at least a portion of a periphery of the chassis forming an upper portion of the first leg opening. The first strap is refastenably engageable with a locking area positioned at the crotch region or the back waist region of the diaper, or both.

Similarly, a second strap, either connected to the first strap or independent of the first strap, is connected to the chassis at the front outer cover of the crotch region or outer cover of the back waist region. The second strap is positionable about at least a portion of the diaper chassis, and is refastenably engageable with a locking area. In one embodiment of this invention, each of the first strap and the second strap include a free end portion forming a locking portion engageable with the locking area. For example, the locking portion of a strap may include a suitable fastener material, such as a hook-type fastener component, at one or both ends of a strap.

One or more locking surfaces or areas for the straps can be positioned with respect to one of the back waist region or the crotch region and are constructed from materials designed to refastenably mate with a strap end. Desirably, the locking area is positioned on one or both of the back waist region and the crotch region on the outer cover. For example, the locking area may be attached or bonded to an exterior surface of the outer cover. Alternatively, the locking area may be integrated with the outer cover.

The mechanism of the present invention may restrict or redirect movement of the diaper with respect to the skin surface of the wearer thereby enabling the regularly designed and placed interior elements of a leak-reduction system, such as containment flaps and leg opening elastics, to operate as normal even though forces placed upon the diaper through wearer movement or exudate loading may change. By applying straps that are engageable with the locking area, the direction of the pulling force on the diaper due to movement or loading is beneficially altered. The straps do not cause any additional stress or discomfort during use. Therefore, red-marking is less likely to occur. The straps are desirably made of a soft feeling material. After the diaper has been soiled, the increase in strain due to the associated increase in weight and expansion of the diaper will be applied to the straps rather than the leg and waist elastics thereby preventing shift of the diaper on the wearer. Because the straps desirably do not elongate under diaper loading, any downward movement of the diaper tending to cause seal failure of the interior leak prevention system will be restricted, thereby helping to maintain the chassis' interior leak-reduction system. As a result, the potential for leakage is reduced.

### BRIEF DESCRIPTION OF THE DRAWINGS

These and other objects and features of this invention will be better understood from the following detailed description taken in conjunction with the drawings, wherein:

FIG. 1 representatively shows an upper right side perspective view of a disposable diaper in a fastened condition with one strap of an exterior leak containment system according to one embodiment of this invention shown fastened on the exterior of the diaper.

FIG. 2 representatively shows a plan view of a disposable diaper in an unfastened, stretched and laid flat condition with a surface of the diaper that contacts the wearer's skin facing away from the viewer and portions of the diaper partially cut away to more clearly show the interior construction of the diaper, according to one embodiment of this invention.

FIG. 3 representatively shows a plan view of a disposable diaper in an unfastened, stretched and laid flat condition with a surface of the diaper that contacts the wearer's skin facing toward the viewer and portions of the diaper partially cut away to more clearly show the interior construction of the diaper, according to one embodiment of this invention;

FIG. 4 representatively shows a plan view of a disposable diaper with two straps positioned at a crotch region;

FIG. 5 representatively shows a strap according to one embodiment of this invention; and

FIGS. 6-9 show side, front, rear and bottom views, respectively, of a garment with the straps in a desirable operational position.

FIG. 10 illustrates a strap formed from, and integral with, the outer cover material of the garment.

### DEFINITIONS

As used herein, the term “bonded” refers to the permanent or semi-permanent joining, adhering, connecting, attaching, or the like, of two elements. Two elements will be considered bonded together when they are bonded directly to one another or indirectly to one another, such as when each is directly bonded to intermediate elements.

As used herein, the term “bonded-carded web” refers to webs that are made from staple fibers which are sent through a combing or carding unit, which separates or breaks apart and aligns the staple fibers in the machine direction to form a generally machine direction-oriented fibrous nonwoven web. Once the web is formed, it then is bonded by one or more of several known bonding methods.

As used herein, the term “comprises,” “comprising,” and other derivatives from the root term “comprise” are intended to be open-ended terms that specify the presence of any stated features, elements, integers, steps or components, but do not preclude the presence or addition of one or more other features, elements, integers, steps, components or groups thereof. Accordingly, such terms are intended to be synonymous with “has,” “have,” “having,” “includes,” “including,” and any derivatives of these words.

As used herein, the term “connected” refers to the joining, adhering, bonding, attaching, or the like, of two elements, whether permanent, semi-permanent or refastenably. Two elements will be considered connected together when they are connected directly to one another or indirectly to one another, such as when each is directly connected to intermediate elements.

As used herein, the term “disposable” refers to articles that are designed to be discarded after a limited use rather than being laundered or otherwise restored for reuse.

As used herein, the terms “elastic,” “elasticized” and “elasticity” refer to a property of a material or composite by virtue of which the material or composite tends to recover its original size and shape after removal of a force causing a deformation. “Extensible” refers to the property of a material or composite by virtue of which the material or composite can extend but not recover its original size and shape after removal of a force causing a deformation.

As used herein, the term “fabrics” is used to refer to all of the woven, knitted and nonwoven fibrous webs.

As used herein, the term “flexible” refers to materials which are compliant and which will readily conform to the general shape and contours of the wearer's body.

As used herein, the term “integral” is used to refer to various portions of a single unitary element rather than separate structures bonded to or placed with or placed near one another.

As used herein, the term “layer” when used in the singular can have the dual meaning of a single element or a plurality of elements.

As used herein, the term “locking area” refers to an area on the outer cover of the diaper engageable with the straps to releasably secure the straps to the outer cover.

As used herein, the terms “longitudinal” and “transverse” or “lateral” have their customary meanings. The longitudinal axis lies in the plane of the article when laid flat and fully extended and is generally parallel to a vertical plane that bisects a standing wearer into left and right body halves when the article is worn. The transverse axis lies in the plane of the article generally perpendicular to the longitudinal axis.

As used herein, the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Such a process is disclosed, for example, in U.S. Pat. No. 3,849,241.

As used herein, the term “member” when used in the singular can have the dual meaning of a single element or a plurality of elements.

As used herein, the term “nonwoven” and “nonwoven web” refer to materials and webs of material that are formed without the aid of a textile weaving or knitting process.

As used herein “pattern unbonded” or “point unbonded” or “PUB,” are used interchangeably herein to mean a fabric pattern having continuous bonded areas defining a plurality of discrete unbonded areas. The fibers or filaments within the discrete unbonded areas are dimensionally stabilized by the continuous bonded areas that encircle or surround each unbonded area, such that no support or backing layer of film or adhesive is required. The unbonded areas are specifically designed to afford spaces between fibers or filaments within the unbonded areas. PUB fabrics are disclosed in U.S. Pat. No. 5,858,515 to Stokes, et al., the disclosure of which is incorporated herein by reference.

As used herein, the term “permanently bonded” refers to the joining, adhering, connecting, attaching, or the like, of two elements of an absorbent article such that the elements tend to be and remain bonded during normal use conditions of the absorbent article.

As used herein, the term “personal care product” includes diapers, training pants, absorbent underpants, adult incontinence products, and feminine hygiene products.

As used herein, the term “refastenable” refers to the property of two elements being capable of releasable attachment, separation, and subsequent releasable reattachment without substantial permanent deformation or other damage to either element.

As used herein, the term “releasably attached,” “releasably engaged,” and variations thereof refer to two elements being connected or connectable such that the elements tend to remain connected absent a separation force applied to one or both of the elements and the elements being capable of separation without substantial permanent deformation or other damage. The required separation force is typically beyond that encountered while wearing the absorbent article.

As used herein, the term “spunbond fibers” refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al.

Words of degree, such as “about”, “substantially”, and the like are used herein in the sense of “at, or nearly at, when given the manufacturing, design, and material tolerances inherent in the stated circumstances” and are used to prevent the unscrupulous infringer from unfairly taking advantage of the invention disclosure where exact or absolute figures are stated as an aid to understanding the invention.

As used herein, “ultrasonic bonding” means a process performed, for example, by passing the fabric between a sonic horn and anvil roll, e.g., as illustrated in U.S. Pat. Nos. 5,552,013 and 5,562,790 to Ehlert et al.

### DESCRIPTION OF PREFERRED EMBODIMENTS

The various aspects and embodiments of the present invention will be described in the context of disposable absorbent articles, and more particularly referred to, without limitation and by way of illustration only, as a disposable diaper. It is apparent that the articles and methods of the present invention are equally adaptable for other types of absorbent articles, such as training pants, adult incontinence articles, personal care garments, and other disposable articles and garments.

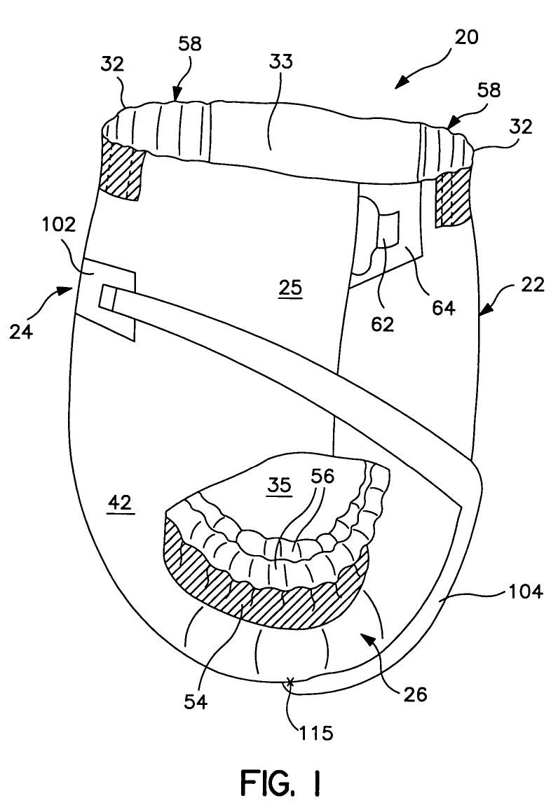

Referencing especially FIGS. 1-3, in one embodiment of this invention, a diaper 20 comprises an exterior leak prevention system including a locking area 102, and first and second straps 104, and 106. Locking areas are desirably positioned at one or more of a crotch region 26 or a back waist region 24 of the diaper 20. The first strap 104 extends from a bond point 115 (FIG. 1) at the crotch region 26 and in use can be placed about a periphery of a chassis, such as along a periphery of a first leg opening 35, across a side panel region 25 and can be refastenably engageable with the locking area 102. A second strap 106 is refastenably engageable with the locking area 102 and extends from the crotch region 26 about a periphery of a second leg opening (also 35), and across the opposite side panel.

The exterior leak prevention system constrains the diaper 20 from sagging or moving downwardly with respect to the body of the wearer, to maintain proper fit about the legs and waist region of the wearer. Unlike the present invention, conventional diapers and absorbent articles sag or move with respect to the wearer's body after the diaper is soiled as a result of the additional weight and expansion of the diaper components, and/or movement of the wearer, thereby displacing the leak control structures of the garment.

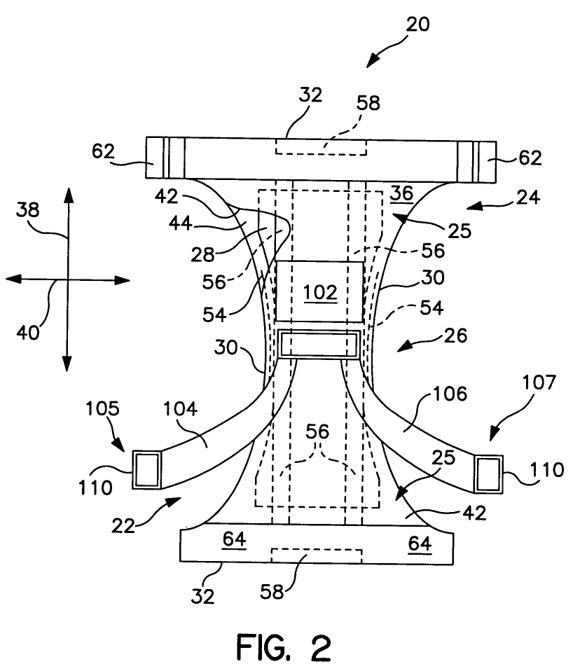

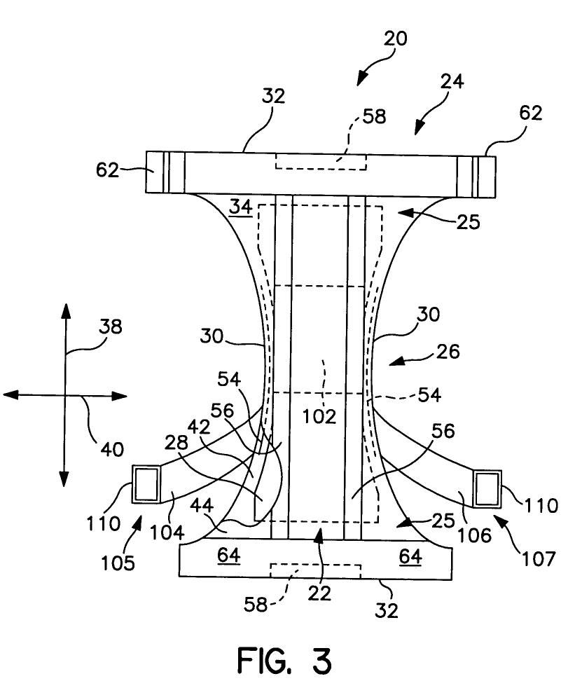

A disposable diaper 20 of the present invention defines a front waist region 22, a back waist region 24, and an intermediate or crotch region 26 that extends between and connects the front waist region 22 and the back waist region 24. For reference, arrows 38 and 40 depicting the orientation of the longitudinal axis and the transverse or lateral axis, respectively, of the diaper 20 are illustrated in FIGS. 2 and 3. In one embodiment of this invention, the crotch region 26 comprises about 50% of the overall length of the diaper 20 in the longitudinal direction 38 and each of the front waist region 22 and the back waist region 24 comprises about 25% of the overall length of the diaper 20 in the longitudinal direction 38, on opposing sides of the crotch region 26.

The front waist region 22 includes the portion of the diaper 20 that, when worn, is positioned on the front of the wearer while the back waist region 24 includes the portion of the diaper 20 that, when worn, is positioned on the back of the wearer. The crotch region 26 of the diaper 20 includes the portion of the diaper 20 that, when worn, is positioned between the legs of the wearer and covers the lower torso of the wearer. Thus, the crotch region 26 is an area where repeated liquid surges typically occur in the diaper 20 or other disposable absorbent articles.

The diaper 20 has a pair of laterally opposing side edges 30, a pair of longitudinally opposing waist edges 32, an interior surface 34 (FIG. 3) that is configured to contact the wearer, and an exterior surface 36 (FIG. 2) that is configured to contact the wearers clothing during use. Referring to FIGS. 1-3, the diaper 20 also includes an outer cover 42 and a bodyside liner 44 that is connected to the outer cover 42 in a superposed relation. An absorbent core 28 is positioned or located between the outer cover 42 and the bodyside liner 44. The outer cover 42, the bodyside liner 44 and the absorbent core 28 define a chassis of the diaper 20, which forms the waist opening 33 and each leg opening 35 when complementary fasteners 62, 64 are used to fasten the diaper 20 about the wearer, as known in the art. The laterally opposing side edges 30 are generally defined by the side edges of the outer cover 42 that further define leg openings 35, which desirably are curvilinear. The waist edges 32 are generally defined by the waist edges of the outer cover 42 and form the waist opening 33 that is configured to encircle the waist of the wearer during use. The absorbent core 28 is configured to contain and/or absorb body exudates discharged from the wearer.

In accordance with one embodiment of this invention, at least a portion of the waist edges 32 and the side edges 30 are elastic or extensible to improve the fit range of the diaper 20 and support the absorbent core 28 to prevent leakage of body exudates and thereby form a portion of the interior leak prevention system of the diaper. For example, referring to FIGS. 1-3, the diaper 20 may include a pair of leg elastic members 54 that are connected to the laterally opposing side edges 30 of the diaper 20 in the crotch region 26. The diaper 20 may also include a pair of waist elastic members 58 that are connected to the longitudinally opposing waist edges 32 of the diaper 20. The leg elastic members 54 and the waist elastic members 58 are generally adapted to fit about the legs and waist of a wearer, respectively, to maintain a positive, contacting relationship with the wearer to effectively reduce the leakage of body exudates from the diaper 20.

Materials suitable for use as the leg elastic members 54 and the waist elastic members 58 are well known to those skilled in the art. For example, sheets, filaments, strands or ribbons of a polymeric, elastomeric material may be adhered to the outer cover 42 in a stretched position or attached to the outer cover 42 while the outer cover 42 is pleated, such that elastic constrictive forces are imparted to the outer cover 42. The leg elastic members 54 may also include such materials as polyurethane, synthetic rubber and natural rubber.

Referring to FIGS. 1-3, the diaper 20 may include a pair of containment flaps 56 that are configured to provide a barrier to the lateral flow of body exudates and which also form a portion of the interior leak prevention system of the diaper 20. The containment flaps 56 may be located along the laterally opposing side edges 30 of the diaper 20 adjacent the side edges of the absorbent core 28. As known, each containment flap 56 typically defines an unattached edge that is configured to maintain an upright, perpendicular configuration in at least the crotch region 26 of the diaper 20 to form a seal against the wearer's body. Each containment flap 56 extends longitudinally along at least a portion of a length of the absorbent core 28. Desirably, each containment flap 56 extends along substantially the entire length of the absorbent core 28 to better contain the body exudates. The containment flaps 56 can be selectively positioned with the attached edge proximate to the side edges 30 of the diaper 20. Such containment flaps 56 are generally well known to those skilled in the art. For example, suitable constructions and arrangements for containment flaps 56 described in U.S. Pat. No. 4,704,116, issued Nov. 3, 1987 to K. Enloe, the disclosure of which is hereby incorporated by reference.

The diaper 20 may be of various suitable shapes. For example, in the unfastened configuration as shown in FIGS. 2, 3 and 4, the diaper 20 may have an overall rectangular shape, T-shape or an approximate hourglass shape. In the shown embodiment, the diaper 20 has a generally I-shape in an unfastened configuration.

The various components of the diaper 20 are integrally assembled together employing various types of suitable attachment means, such as adhesive, sonic and thermal bonds, and combinations thereof. In the shown embodiment, for example, the outer cover 42 and bodyside liner 44 are assembled to each other and to the absorbent core 28 with an adhesive, such as a hot melt, pressure-sensitive adhesive. The adhesive may be applied as a uniform continuous layer of adhesive, a patterned layer of adhesive, a sprayed pattern of adhesive, or an array of separate lines, swirls or dots of adhesive. Similarly, the other components of the diaper 20 may be assembled into the diaper 20 by employing the above-identified attachment mechanisms.

The outer cover 42 of the diaper 20 may suitably be composed of a material that is either water vapor permeable or water vapor impermeable. The outer cover material may be extensible, elastic, or non-extensible, as needed, taking into consideration the amount of extension of the outer cover which may tolerated under loading of the diaper according to the teachings of the present invention. It is generally desired that the outer cover 42 be formed from a material that is substantially impermeable to liquids and permeable (breathable) to water vapor. The outer cover 42 can be manufactured from a thin plastic film or other suitable flexible liquid-impermeable material. For example, the outer cover 42 may be formed from a polyethylene film having a thickness of about 0.013 millimeter (0.5 mil) to about 0.051 millimeter (2.0 mils). If a more clothlike feeling is desired, the outer cover 42 may be formed from a polyolefin film having a nonwoven web, such as a spunbond web of polyolefin fibers, laminated to an exterior surface thereof.

The bodyside liner 44 suitably presents a bodyfacing surface that is compliant, soft feeling, and nonirritating to the wearer's skin. Suitable web materials for manufacturing the bodyside liner 44 include, but are not limited to, porous foams, reticulated foams, apertured plastic films, natural fibers (for example, wood or cotton fibers), synthetic fibers (for example, polyester or polypropylene fibers), and/or a combination of natural and synthetic fibers. The bodyside liner 44 is suitably employed to help isolate the wearer's skin from liquids held in the absorbent core 28.

Various woven and nonwoven fabrics can be used for the bodyside liner 44. For example, the bodyside liner 44 may be composed of a meltblown or spunbond web of polyolefin fibers. The bodyside liner 44 may also be a bonded-carded web composed of natural and/or synthetic fibers. The absorbent core 28, desirably composed of selected hydrophilic fibers and high-absorbency particles, is positioned or located between the bodyside liner 44 and the outer cover 42. The absorbent core 28 desirably has a construction that is generally compressible, conformable, non-irritating to the wearer's skin, and capable of absorbing and retaining body exudates. It should be understood that the absorbent core 28 may comprise a single, integral piece of material, or alternatively, may comprise a plurality of individual separate pieces of material, which are assembled together.

The absorbent core 28 of the diaper 20 may suitably be composed of a matrix of hydrophilic fibers, such as a web of cellulosic fluff, mixed with particles of a high-absorbency material commonly known as superabsorbent material. In one embodiment of this invention, the absorbent core 28 includes a matrix of cellulosic fluff such as wood pulp fluff and superabsorbent hydrogel-forming particles. The wood pulp fluff may be exchanged with synthetic, polymeric, meltblown fibers, or with a combination of meltblown fibers and natural fibers. The superabsorbent particles may be substantially homogeneously mixed with the hydrophilic fibers or may be nonuniformly mixed. The fluff and superabsorbent particles may also be selectively placed into desired zones of the absorbent core 28 to better contain and absorb body exudates. The concentration of the superabsorbent particles may also vary through the thickness of the absorbent core 28. Alternatively, the absorbent core 28 may comprise a laminate of fibrous webs and superabsorbent material or other suitable means of maintaining a superabsorbent material in a localized area. A substantially hydrophilic tissue or nonwoven wrapsheet (not shown) may be employed to help maintain the integrity of the airlaid fibrous structure of the absorbent core 28.

The diaper 20 further comprises the exterior leak prevention system including a locking surface, or area, 102 shown as positioned with respect to the medial area of the rear waist section 24. Locking areas may be located at any or all areas of the back waist region 24 or the crotch region 26 or the side panel regions 25. Desirably, the locking area 102 is positioned with respect to either the crotch region 26 or the back waist region 24. The locking area 102 desirably is refastenably engageable with at least one strap, for example strap 104, to maintain the diaper 20 in proper position about wearer, particularly after the diaper 20 has been soiled.

Desirably, a locking area 102 located in the medial area of the rear waist section 24, or elsewhere on the diaper 20, has a length in the longitudinal direction 38 and a width in the transverse direction 40 sufficient to accommodate a range of placements of the free ends 105, 107 of the straps 104, 106 in keeping with the size and shape of the individual wearer.

In one embodiment of this invention, the locking area 102 may be integrated with the outer cover 42 at the exterior surface 36 of the diaper 20 by utilizing a fabric for the outer cover which can accept the fastener of the strap, or by using a large panel of material for the locking area affixed to the outer cover, e.g., a so-called PUB fabric acting as the loop half of a hook and loop fastener to which the first strap 104 and the second strap 106 are releasably engageable. Alternatively, locking areas can be attached or bonded to the outer cover 42 of the diaper 20 in one or more sections to accommodate one or both ends of the straps 104, 106. The locking area 102 is made of a suitable material that is engageable with a complementary fastener system 110 at the free ends 105, 107 of the first strap 104 and the second strap 106 as further described below. Other suitable materials for the locking area 102 may include, but are not limited to, adhesives, co-adhesives, and other known fastening systems deemed suitable for the task.

Referring to FIGS. 1-4, the exterior leak prevention system desirably further includes at least one strap, e.g. 104, attached or connected at a first end with respect to the chassis, and having a second end with a free end portion 105, 107 having a cooperative fastener, collectively 110, for refastenably engaging with the locking area 102. As shown in FIGS. 2 and 3, a first strap 104 and a second strap 106 each are connected or bonded to the chassis and refastenably engageable with the locking area 102 at a free end portion 105 and 107, respectively. The first strap 104 and the second strap 106 may be bonded, e.g., adhesively, to the chassis in the back waist region 24 or the crotch region 26. Alternatively, the straps may be formed integrally from the outer cover material as described below with respect to FIG. 10. Desirably, each of the first strap 104 and the second strap 106 are positioned with respect to the crotch region 26, proximal to the lateral side edges 30 and overlaying the containment flaps 56 located on the interior of the diaper, as shown in FIG. 2.

As illustrated, the first strap 104 and the second strap 106 each are attached or bonded, e.g. adhesively, to the outer cover 42 of the diaper at the crotch region 26, exclusively, i.e. the attachment areas of a first end of the first strap 104 and the second strap 106 do not extend into the front waist region 22 and/or the back waist region 24. As discussed above, the crotch region 26 comprises about the middle 50% of the longitudinal length of the diaper 20, with the front waist region 22 and the back waist region 24 each comprising about 25% of the longitudinal length of the diaper 20. It will be noted, however, that the first strap 104 and the second strap 106 can be attached or bonded to the back waist region 24, and left with a free end to be attached in the crotch area, in alternative embodiments of this invention.

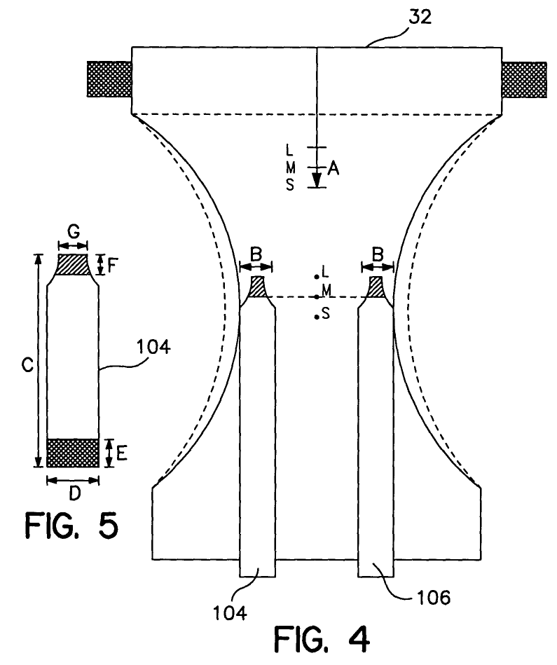

Referencing FIG. 4, each of the first strap 104 and the second strap 106 may have a suitable length to engage the locking area 102 and redistribute the force of a loaded diaper to maintain the diaper 20 in proper position about the wearer's waist and prevent downward movement of the diaper 20 with respect to the wearer's waist after the diaper 20 has been soiled. By way of example, for a HUGGIES Step 3 size diaper, the first strap 104 and the second strap 106 may have a length in the longitudinal direction 38 of about 19 cm to about 31 cm, and have a width in the transverse direction 40 of about 3 cm, as further discussed below. It is apparent that the first strap 104 and the second strap 106 each will have suitable dimensions, depending, for example, upon the size of the intended wearer and the amount of force exerted on the straps 104 and 106 after the diaper 20 has been soiled.

The straps 104, 106 are desirably made from a material which does not tension the diaper upon donning and dry wear of the garment but which provides tensioning of the garment upon loading of the diaper to support the garment, per the present discussion. For example, straps may be made of 1.8 osy basis weight nonwoven material laminate of the spunbond/meltblown/spunbond (SMS) type, as known in the art. The first strap 104 and the second strap 106 are desirably made of a non-extensible material, such as a nonwoven having suitable hand and appearance for the present purpose. Other suitable materials may include, but are not limited to, various woven fabrics or films of natural or man-made material as deemed desirable. Alternatively, the strap material may be extensible or elastic, as needed, taking into consideration the amount of extension of the straps which may tolerated under loading of the diaper according to the teachings of the present invention.

The locking area 102 is positioned to be refastenably engageable with the first strap 104 and the second strap 106 and desirably, to follow a portion of the containment flap 56 or leg elastic 54 placement in the crotch area 26 to maintain the interior leak prevention system of the diaper 20 in proper position and in contact with the skin surface of the legs. Thus, leakage of bodily exudates from the leg openings 35 of the diaper 20 is prevented under the strain of a loaded diaper.

Without being bound by theory, the exterior leak prevention system prevents leakage of bodily exudates from the leg openings 35 by changing the direction of force against the chassis associated with the increased weight and expansion of the diaper 20 after the diaper 20 has been soiled. The leak prevention system constrains the diaper 20 from pulling away from the legs of the wearer as a result of the downward movement of the diaper 20, as is typically seen in conventional diapers and absorbent articles. Because the first strap 104 and the second strap do not elongate under loading and are supported at the leg opening areas, the force exerted on the first strap 104 and the second strap 106 as a result of soiling the diaper 20 does not cause any downward movement of the diaper 20 with respect to the wearer's leg region. Thus, the diaper 20 remains in proper position and spaces do not form between the interior leakage containment system and the skin surface of the wearer.

### EXAMPLES

Testing was done on three types, or codes, of commercial diapers having an interior containment system including containment flaps experimentally modified to include an exterior containment system according to the present invention. The modified diapers are compared to an unmodified control diaper of the same commercial type without straps. Referring also to FIG. 4, wherein relative distances of attachment are indicated along the longitudinal axis of the garment, Diaper Code M had strap first ends attached at about the middle of the laid flat diaper in the longitudinal direction, i.e., in the center of the crotch region. Diaper Code L had long strap first ends attached about 2 cm away from the center of the crotch region towards the back waist margin. Diaper Code S had short strap first ends attached about 2 cm away from the center of the crotch region towards front waist margin. Actual strap dimensions are set forth below. The diapers were leak tested on a mannequin in a sitting position and a combination of sitting and walking positions, as set forth below.

Sitting Position Test

The diaper to be tested is weighed and put on a mannequin and the mannequin is made to sit with the legs at about a 90° angle to the trunk. Then fluid is introduced to the interior of the diaper in the following format:
1. Insult diaper with 60 ml fluid, fluid flow at the rate of 10 mL/sec
2. Wait 15 min.
3. Repeat steps 3 and 4 until the diaper leaks.
4. Stop the pump at the first sight of leak.

Sitting and Walking Tests

In these tests, the mannequin is made to simulate walking by moving both the legs back and forth, while keeping the trunk stable. In addition the diaper is tested in the sitting position. The fluid introduction is in the sitting position. The test protocols are as follows.

Version 1:
1. Take dry diaper weight.
2. Apply diaper to mannequin.
3. Walk Mannequin at a pace of 50 steps/min for a period of 5 minutes before insult.
4. Insult diaper in sitting position w/60 ml saline dyed blue for visual assessment of leaks at a rate of 10 mL/sec.
5. Allow mannequin to sit for 30 seconds.
6. Walk mannequin for 5 min.
7. Let mannequin stand for 10 min.
8. Repeat steps 4 through 7 until leak.
9. Weigh wet diaper.

Version 2:
1. Take dry diaper weight.
2. Apply diaper to mannequin.
3. Walk mannequin 5 minutes at a pace of 50 steps/min before insult.
4. Insult diaper in sitting position w/60 ml saline dyed blue for visual assessment of leaks at a rate of 10 mL/sec.
5. Allow mannequin to sit for 30 seconds.
6. Walk mannequin for 1 min.
7. Let mannequin stand for 4 min.
8. Repeat steps 4 through 7 until leak.

9. Weigh wet diaper.

TABLE 1
Sitting test results

| | Amount of |
|---|---|
| CODE | fluid retained, g |
| Control-1 | 230.93 |
| Control-2 | 175.24 |
| Control-3 | 118.09 |
| Control-4 | 227.84 |
| Control-5 | 175.35 |
| Control-6 | 59.35 |
| Control-7 | 119.78 |
| Control-8 | 119.16 |
| Control-9 | 173.92 |
| Control-10 | 176.58 |
| Control-11 | 119.29 |
| Control-12 | 176.94 |
| Control-13 | 178.14 |
| Ave. | 157.74 |
| M1 | 175.42 |
| M2 | 235.25 |
| M3 | 233.15 |
| M4 | 177.04 |
| M5 | 117.99 |
| M6 | 176.5 |
| M7 | 117.99 |
| M8 | 117.94 |
| M9 | 117.4 |
| M10 | 234.17 |
| M11 | 174.18 |
| M12 | 117.56 |
| M13 | 115.07 |
| Ave. | 162.28 |
| L1 | 118.41 |
| L2 | 118.35 |
| L3 | 177.05 |
| L4 | 113.02 |
| L5 | 173.52 |
| Ave. | 140.07 |
| S1 | 118.94 |
| S2 | 120.27 |
| S3 | 117.31 |
| S4 | 116.81 |
| S5 | 174.4 |
| Ave. | 129.55 |

TABLE 2
Sitting and walking test results, version 1

| | Amount of |
|---|---|
| CODE | fluid retained, g |
| Control-14 | 102.72 |
| Control-15 | 173.35 |
| Control-16 | 175.06 |
| Control-17 | 176.07 |
| Control-18 | 176.35 |
| Control-19 | 170.95 |
| Control-20 | 175.65 |
| Control-21 | 175.24 |
| Control-22 | 173.75 |
| Control-23 | 173.21 |
| Ave. | 167.24 |
| M14 | 176.74 |
| M15 | 117.11 |
| M16 | 116.1 |
| M17 | 231.35 |
| M18 | 169.64 |
| M19 | 232.83 |
| M20 | 177.35 |
| M21 | 176.69 |
| M22 | 178.13 |
| M23 | 118.09 |
| Ave. | 169.40 |

TABLE 3
Sitting and walking test, version 2

| Code | Amount of fluid retained, g | Average |
|---|---|---|
| C1 | 175.49 | |
| C2 | 118.19 | |
| C3 | 116.36 | |
| C4 | 117.55 | |
| C5 | 117.99 | |
| C6 | 60.55 | |
| C7 | 125.81 | |
| C8 | 47.6 | |
| C9 | 59.75 | |
| C10 | 118.85 | 105.81 |
| L1 | 119.96 | |
| L2 | 120.46 | |
| L3 | 119.56 | |
| L4 | 117.72 | |
| L5 | 120.53 | |
| L6 | 120.21 | |
| L7 | 117.65 | |
| L8 | 117.32 | |
| L9 | 117.27 | |
| L10 | 60.83 | 113.15 |
| M1 | 120.38 | |
| M2 | 120.26 | |
| M3 | 119.42 | |
| M4 | 179.27 | |
| M5 | 119.72 | |
| M6 | 120.06 | |
| M7 | 120.22 | |
| M8 | 121.28 | |
| M9 | 61 | |
| M10 | 120.43 | 120.20 |
| S1 | 120.19 | |
| S2 | 120.41 | |
| S3 | 119.54 | |
| S4 | 119.66 | |
| S5 | 119.65 | |
| S6 | 119.71 | |
| S7 | 119.82 | |
| S8 | 119.7 | |
| S9 | 119.85 | |
| S10 | 119.92 | 119.85 |

In all cases, the total amount of fluid retained was measured by the difference in weight of the diaper before and after the test. Fluid delivery was by a peristaltic pump.

The dimensions of a standing Standard Step 3 size Unisex mold mannequin as used for the tests are as follows:
1) 17″ or 43 cm. Waist circumference at navel: (hold measuring tape parallel to ground)
2) 18″ or 46 cm. Hip circumference at pelvic bone: (hold measuring tape parallel to ground)
3) 18.5″ or 47 cm. Hip/Seat at widest point around buttocks: (hold measuring tape parallel to ground)
4) 12″ or 31 cm. Pant/diaper line: thigh circumference along pant-line (natural body line crease)
5) 10.5″ or 27 cm. Thigh circumference: upper thigh (hold measuring tape parallel to ground)
6) 11.5″ or 29 cm. Rise: measure from navel down between legs and up to small of the back
7) 11.5″ or 29 cm. Height of Torso: from waist to knee (hold measuring tape perpendicular to ground)

Referring to FIGS. 4 and 5, as well as Tables 4 and 5 below, for dimension codes A-G of the experimental diapers, the defined dimensions of the exterior containment system for the test diapers were as follows. The variable Dimension “A” is the longitudinal distance from the back end waist margin 32 of the diaper to the point of strap attachment. Dimension “B” is the maximum distance from the lateral side margin within which the strap is attached. Dimension “C” is the total length of the strap. Dimension “D” is the width of the strap. Dimension “E” is the length of the refastenable area. Dimension “F” is the length of the adhesive area (that is attached near position “B”). Dimension “G” is the width of the narrowest end of that attachment.

TABLE 4
Strap Dimensions in Sitting Test

| Code | A: | B | C | D | E | F | G |
|---|---|---|---|---|---|---|---|
| L | 17 cm | Up to 7 cm | 31 cm | 3 cm | 2.5 cm | 2.5 cm | 3 cm |
| M | 21 cm | Up to 7 cm | 27 cm | 3 cm | 2.5 cm | 2.5 cm | 3 cm |
| S | 25 cm | Up to 7 cm | 23 cm | 3 cm | 2.5 cm | 2.5 cm | 3 cm |

| | |
|---|---|
| Con- | Huggies Supreme, Step 3 (commercial product) |
| trol | |

TABLE 5
Strap Dimensions in Sitting and Walking Tests

| Code | A: | B | C | D | E | F | G |
|---|---|---|---|---|---|---|---|
| L | 17 cm | 2 cm | 23.5 cm | 3 cm | 3 cm | 2 cm | 1.5 cm |
| M | 21 cm | 2 cm | 21.5 cm | 3 cm | 3 cm | 2 cm | 1.5 cm |
| S | 25 cm | 2 cm | 19.5 cm | 3 cm | 3 cm | 2 cm | 1.5 cm |

| | |
|---|---|
| Control | Huggies Supreme, Step 3 (commercial product) |

All straps were attached proximate to the side edges. There was some variation in the distance from the side edge to the strap in placement on individual diapers.

In all cases, the experimental diapers, i.e., codes S, M and L, utilized the same type diapers as that of the controls. The straps were made of 1.8 osy SMS material. One end of each strap was permanently attached to the outer cover of the diaper using adhesive as indicated by the darkened areas near the markings “B”, “F” and “G” of FIGS. 4 and 5. The free end of each strap, as indicated by the darkened area near markings “D” and “E” of FIGS. 4 and 5, was attached to the diaper using hook & loop mechanisms.

The test results indicate that the addition of the straps reduces leakage. Variations in the test data may be due to the variation in the position of attachment in the diaper transverse direction (the “B” measurement). When the straps are placed closer to the edge (lower “B” value), the gaskets of the diaper are kept in place, close to the body, even when the diaper is filled with fluid. This is due to the support of the added straps. On the other hand, when the straps are attached at a position farther away from the edges (larger “B” value), the added straps are not necessarily supporting the gaskets on the side. Conversely, the straps prevent the diaper from expanding at the middle. This reduces the space available for liquid to flow within the crotch area and allows liquid to flow out of the diaper.

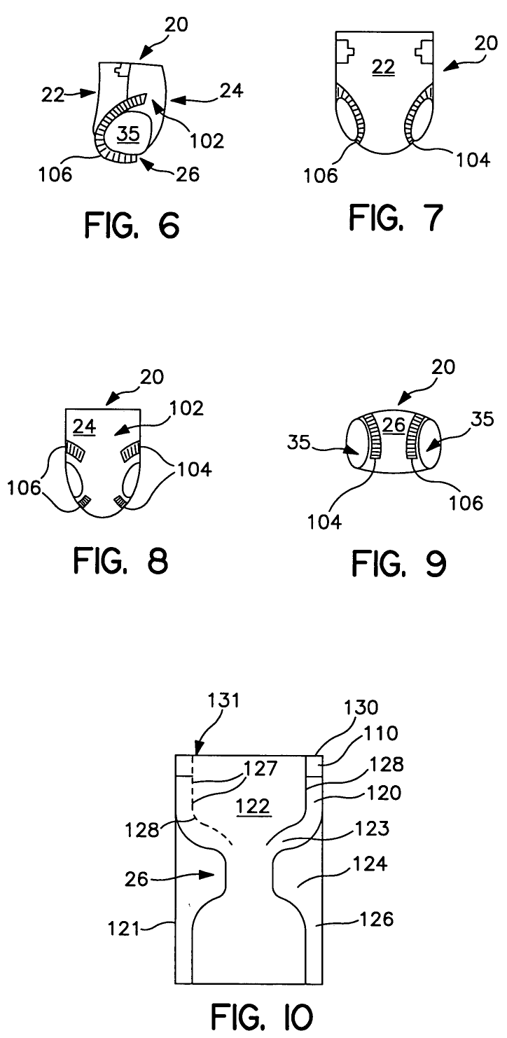

FIGS. 6-9 show side, front, rear and bottom views, respectively, of a garment with the straps 104, 106 in a desirable operational position. The first strap 104 is bonded to the outer cover of the garment 20 at a first end thereof and is refastenably engaged with the locking area 102 at a second end thereof. The first strap 104 extends from the crotch region 26 about a periphery of a leg opening 35 of the garment 20 to the back waist region 24 on a first side of the garment 20. The second strap 106 is bonded to the outer cover of the garment 20 at a first end thereof and refastenably engaged with the locking area 102 at a second end thereof. The second strap 106 extends from the crotch region 26 about a periphery of a leg opening 35 of the garment to the back waist region 24 of the garment on a second side of the garment.

Referring to FIG. 10, according to another aspect of the present invention, an integral strap 120 may be formed from a web 121 of outer cover material 122 by selective cutting of the outer cover material 122, thereby leaving a strap first end 123 extending seamlessly from the outer cover material 122 at a lateral edge of the crotch area 26. The cutting may be done at any convenient time during the garment forming process. For example, the integral strap 120 may comprise the outer cover material 122 alone, or may comprise the outer cover material 122 and various materials connected thereto. In one aspect, the so-called leg cut-out 124 normally formed in the web 121 of outer cover material 122 may be formed in the outer cover material 122 at substantially the same time as the integral strap 120 is formed. The excess material 126 may be clear cut for removal from the web of outer cover material 122 while a cut 128 for formation of the strap 120 may comprise perforations 127 forming a line of weakness 131 in the outer cover material 122 to aid in maintaining the stable processing and handling of the outer cover material 122 during garment formation. It will be appreciated that a fastener 110 may need to be bonded to the second end 130 of the integral strap 120 in certain embodiments.

While in the foregoing specification this invention has been described in relation to certain preferred embodiments thereof, and many details have been set forth for purpose of illustration, it will be apparent to those skilled in the art that the invention is susceptible to additional embodiments and that certain of the details described herein can be varied considerably without departing from the basic principles of the invention.

1. A disposable garment comprising:
a chassis with an interior leak prevention system having at least one of leg elastics, waist elastics, and containment flaps; and
an exterior leak prevention system having a strap connected on, or formed integrally with, an exterior surface of the garment at the crotch region of the garment and connectable at a second point on the exterior surface of the garment to support additional load on the garment when the garment is loaded.

2. The disposable garment of claim 1 wherein the strap comprises a non-extensible material.

3. The disposable garment of claim 1 wherein the strap is refastenably connectable to at least one point on the exterior surface

4. The disposable garment of claim 1 wherein the exterior leak prevention system further comprises a first strap and a second strap.

5. The disposable garment of claim 1 wherein the strap is positionable between a first point at the crotch region of the garment and a second point at one of a back waist region and a side panel region of the garment.

6. The disposable garment of claim 1 wherein the interior leak prevention system comprises containment flaps and the straps are positionable to follow a position of the containment flaps when the garment is donned by a wearer.

7. A disposable garment comprising:
a chassis with an interior leak prevention system having at least one of leg elastics, waist elastics, and containment flaps; and
an exterior leak prevention system having a strap connected on an exterior surface of the garment at the crotch region of the garment and connectable at a second point on the exterior surface of the garment to redirect additional load on the article when the article is loaded whereby the interior leak prevention system is maintained in contact with a wearer of the garment.

8. The disposable garment of claim 7 wherein the interior leak prevention system comprises containment flaps.

9. A disposable absorbent article comprising:
a chassis having a front waist region, a back waist region, and left and right side panel regions, and a crotch region intermediate the front waist region and the back waist region, and further including an outer cover;
the chassis forming a waist opening, a first leg opening and a second leg opening;
a locking area positioned on the outer cover;
a first strap connected to, or integral with, the outer cover at the crotch region in a first location apart from the locking area, the first strap positionable over a portion of the chassis, and refastenably connectable to the locking area without creating tension on the chassis; and
a second strap connected to, or integral with, the outer cover at the crotch region in a second location apart from the locking area, the second strap positionable over a portion of the chassis, and refastenably connectable to the locking area without creating tension on the chassis.

10. The disposable absorbent article of claim 9 wherein each of the first and second locations apart from the locking area are located at the crotch region of the article proximate to side edges of the article.

11. The disposable absorbent article of claim 10 wherein the locking area is located in a medial area of a back waist region of the article.

12. The disposable absorbent article of claim 11 wherein the first strap extends across the right side panel region proximate to an upper portion of the first leg opening and the second strap extends across the left side panel region proximate to an upper portion of the second leg opening.

13. The disposable absorbent of claim 9 wherein the locking area is integrated with the outer cover.

14. The disposable absorbent article of claim 9 wherein the locking area is bonded to the outer cover.

15. The disposable absorbent article of claim 9 wherein the first strap and the second strap each comprise a locking portion engageable with the locking area.

16. The disposable absorbent article of claim 15 wherein the strap comprises a non-extensible material.

17. A leak prevention system for an absorbent article, the leak prevention system comprising:
one or more locking areas positioned at a back waist region of the absorbent article;
a first strap bonded at a first end thereof to an outer cover of the garment at a crotch region of the garment, and refastenably engageable with the one or more locking areas at a second end thereof;
the first strap extendable from the crotch region about a periphery of a first leg opening of the article to the one or more locking areas on a first side of the article;
a second strap bonded at a first end thereof to an outer cover of the article at the crotch region of the garment, and refastenably engageable with the one or more locking areas at a second end thereof;
the second strap extendable from the crotch region about a periphery of a second leg opening of the article to the one or more locking areas on a second side of the article.

18. A leak prevention system for an absorbent article, the leak prevention system comprising:
one or more locking areas positioned at a crotch region of the absorbent article;
a first strap bonded at a first end thereof to an outer cover of the garment at a back waist region of the garment, and refastenably engageable with the one or more locking areas at a second end thereof;
the first strap extendable from the back waist region about a periphery of a first leg opening of the article to the one or more locking areas on a first side of the article;
a second strap bonded at a first end thereof to an outer cover of the article at a back waist region of the garment, and refastenably engageable with the one or more locking areas at a second end thereof;
the second strap extendable from the back waist region about a periphery of a second leg opening of the article to the one or more locking areas on a second side of the article.

19. A leak prevention system for an absorbent article, the leak prevention system comprising:
one or more locking areas positioned at one or more side panel regions of the absorbent article;
a first strap bonded at a first end thereof to an outer cover of the garment at a crotch region of the garment, and refastenably engageable with the one or more locking areas at a second end thereof;
the first strap extendable from the crotch region about a periphery of a first leg opening of the article to the one or more locking areas on a first side of the article;
a second strap bonded at a first end thereof to an outer cover of the article at the crotch region of the garment, and refastenably engageable with the one or more locking areas at a second end thereof;
the second strap extendable from the crotch region about a periphery of a second leg opening of the article to the one or more locking areas on a second side of the article.

20. A disposable garment comprising:
a chassis with an interior leak prevention system having leg elastics; and
an exterior leak prevention system having a strap integrally formed from an outer cover of the garment at the crotch region of the garment and connectable at a second point on the exterior surface of the garment to redirect additional load on the article when the article is loaded whereby the interior leak prevention system is maintained in contact with a wearer of the garment.

21. A leak prevention system for an absorbent article, the leak prevention system comprising:
one or more locking areas positioned at a back waist region of the absorbent article;
a first strap extending integrally from an outer cover of the garment at a first end thereof at a crotch region of the garment, and refastenably engageable with the one or more locking areas at a second end thereof;
the first strap extendable from the crotch region about a periphery of a first leg opening of the article to the one or more locking areas on a first side of the article;
a second strap extending integrally from an outer cover of the garment at a first end thereof at a crotch region of the garment, and refastenably engageable with the one or more locking areas at a second end thereof;
the second strap extendable from the crotch region about a periphery of a second leg opening of the article to the one or more locking areas on a second side of the article.

22. A leak prevention system for an absorbent article, the leak prevention system comprising:
one or more locking areas positioned at one or more side panel regions of the absorbent article;
a first strap extending integrally from an outer cover of the garment at a first end thereof at a crotch region of the garment, and refastenably engageable with the one or more locking areas at a second end thereof;
the first strap extendable from the crotch region about a periphery of a first leg opening of the article to the one or more locking areas on a first side of the article;
a second strap extending integrally from an outer cover of the garment at a first end thereof at a crotch region of the garment, and refastenably engageable with the one or more locking areas at a second end thereof;
the second strap extendable from the crotch region about a periphery of a second leg opening of the article to the one or more locking areas on a second side of the article.
